(19) United States
(12) Patent Application Publication
(10) Pub. No.: US 2021/0228751 A1
(43) Pub. Date: Jul. 29, 2021

(54) Method and Apparatus for Self-Sterilizing a Bottle

(71) Applicants: Nouri Edward Hakim, Monroe, LA (US); Jacob M. Sclare, Calhoun, LA (US)

(72) Inventors: Nouri Edward Hakim, Monroe, LA (US); Jacob M. Sclare, Calhoun, LA (US)

(21) Appl. No.: 16/847,390

(22) Filed: Apr. 13, 2020

Related U.S. Application Data
(60) Provisional application No. 62/966,620, filed on Jan. 28, 2020.

(51) Int. Cl. A61L 2/12; A61J 9/08; A61J 11/00

(52) U.S. Cl. CPC A61L 2/12; A61L 2202/23; A61J 11/0085; A61J 9/085

(57) ABSTRACT

The present invention overcomes the disadvantages of the prior art and provides for an improved method and apparatus for self-sterilizing a bottle. A self-sterilizing bottle comprises a container, a mouthpiece, a collar and a cap. The container has a sidewall and a bottom defining a liquid holding space, with the sidewall forming an open end with a neck. The collar has a first position and a second position and is adapted to fasten to the neck and for removably fastening the mouthpiece to the collar when in the first position. The cap is configured to have an upright position and an inverted position and when in the upright position is adapted to fasten to the collar and cover the mouthpiece when the mouthpiece and cap are attached to the collar and when in the inverted position to rest stably on a surface. Also when in the inverted position, the cap is further adapted to hold a liquid and support the collar above the liquid when the collar is in the second position.

In another embodiment, a method for self-sterilizing a bottle is described wherein the bottle comprises a cap, a collar, a mouthpiece and a container having an open end. The method comprises placing the cap on a support surface in a position to hold a liquid. Then adding a liquid to the cap. Then placing the collar on the cap so that the collar remains above the liquid in the cap. Then placing the mouthpiece within the container. Then placing the open end of the container with the mouthpiece therein on the collar to form a self-sterilizing assembly. Then placing the self-sterilizing assembly in a microwave oven. Then heating the self-sterilizing assembly in a microwave oven for a time sufficient to sterilize the self-sterilizing assembly.

This application claims the benefit of U.S. provisional patent application Ser. No. 62/966,620, filed Jan. 28, 2020, which is incorporated herein by reference in its entirety.

### FIELD OF THE DISCLOSURE

The present disclosure relates generally to bottles and, more particularly, to a method and apparatus for self-sterilizing a bottle.

### BACKGROUND

Nursing bottles used with infants need to be sterile before use. With our increasingly mobile society, it becomes necessary to clean and sterilize a nursing bottle away from home. Conventional nursing bottles and their components generally have to be individually cleaned and separately sterilized, typically using a dishwasher. A problem arises when the nursing bottle needs to be cleaned and sterilized when there is no access to a dishwasher or other appliance typically used for washing and sterilizing bottles. One proposed solution is disclosed in U.S. Pat. No. 8,703,052. That patent describes a method for use with a special bottle that comes apart. That method has a number of disadvantages such as providing a bottle more prone to leak during normal operation. Also the specialized bottle described by the prior art requires complex disassembly steps that make its use difficult for the ordinary consumer. A need has arisen for an improved method and apparatus for self-sterilizing a bottle.

### SUMMARY

The present invention overcomes the disadvantages of the prior art and provides for an improved method and apparatus for self-sterilizing a bottle. A self-sterilizing bottle comprises a container, a mouthpiece, a collar and a cap. The container has a sidewall and a bottom defining a liquid holding space, with the sidewall forming an open end with a neck. The collar has a first position and a second position and is adapted to fasten to the neck and for fastening the mouthpiece to the collar when in the first position. The cap is configured to have an upright position and an inverted position and when in the upright position is adapted to fasten to the collar and cover the mouthpiece when the mouthpiece and cap are attached to the collar and when in the inverted position to rest stably on a surface. Also when in the inverted position, the cap is further adapted to hold a liquid and support the collar above the liquid when the collar is in the second position. In another embodiment, the self-sterilizing bottle further comprises a vent straw. In another embodiment, the self-sterilizing bottle further comprises a vent straw having a reservoir end and a distal end and is adapted to support a mouthpiece at the distal end when the vent straw is supported by the cap. In another embodiment, the self-sterilizing bottle further comprises a vent assembly. In another embodiment, the self-sterilizing bottle further comprises a vent assembly wherein the vent assembly has a vent straw with a distal end and is adapted to support a mouthpiece at the distal end when the vent assembly is supported by the cap. In another embodiment, the cap is adapted to hold 20 ml of liquid when in the inverted position. In another embodiment, the cap has indicia indicating an optimum amount of liquid for self-sterilization. In another embodiment, the mouthpiece and cap are constructed of a flexible material. In another embodiment, the collar and container are constructed of a rigid material.

In another embodiment, a method for self-sterilizing a bottle is described wherein the bottle comprises a cap, a collar, a mouthpiece and a container having an open end. The method comprises placing the cap on a support surface in a position to hold a liquid. Then adding a liquid to the cap. Then placing the collar on the cap so that the collar remains above the liquid in the cap. Then placing the mouthpiece within the container. Then placing the open end of the container with the mouthpiece therein on the collar to form a self-sterilizing assembly. Then placing the self-sterilizing assembly in a microwave oven. Then heating the self-sterilizing assembly in a microwave oven for a time sufficient to sterilize the self-sterilizing assembly. In another embodiment, the liquid of the self-sterilizing assembly is heated to 150° F. and held at or above that temperature for a time sufficient to sterilize the self-sterilizing assembly. In another embodiment, the liquid of the self-sterilizing assembly is at least 20 ml. In another embodiment, the liquid of the self-sterilizing assembly is water.

In another embodiment, a method for self-sterilizing a bottle comprises the following steps. Providing a bottle having a container with a closed end and an open end, a collar, a mouthpiece and a cap having an open end and closed end. Placing the closed end of the cap on a support surface. Adding a liquid to the cap. Placing the collar on the cap so that the collar is above the liquid. Placing the mouthpiece inside the container. Placing the open end of the container, with the mouthpiece, on the open end of the cap to form a self-sterilizing assembly. Placing the self-sterilizing assembly in a microwave oven. Heating the liquid in the self-sterilizing assembly to a temperature of 150° F. and holding the liquid temperature at or above 150° F. for a time sufficient to sterilize the self-sterilizing assembly. In another embodiment, the liquid is water. In another embodiment, the bottle further comprises a vent straw and further comprises the step of supporting the mouthpiece by the vent straw within the container. In another embodiment, the mouthpiece and cap are formed of a flexible material. In another embodiment, the cap further comprises indicia indicating an optimum liquid fill line and the method step of adding the liquid to the level of the indicia. In another embodiment, the container and collar are constructed of a non-flexible material. In another embodiment, the collar forms a seal with the cap.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a view of a self-sterilizing bottle illustrating an apparatus useful in the present invention;

FIG. 2 is an exploded view of the of the self-sterilizing bottle shown in FIG. 1; and

FIG. 3 is a view of the self-sterilizing bottle shown in FIGS. 1 and 2 adapted for use in the method of the present invention.

### DETAILED DESCRIPTION OF THE EMBODIMENTS

This application is related to U.S. provisional patent applications Ser. No. 62/966,615, filed Jan. 28, 2020, for No Colic Bottle and Ser. No. 62/966,617, filed Jan. 28, 2020, for Ergonomic Bottle, and U.S. non-provisional patent applications serial number ______, filed ______, for No Colic Bottle and serial number ______, filed ______, for Ergonomic Bottle, which are incorporated herein by reference in their entirety.

Reference is now made in detail to the description of the embodiments as illustrated in the drawings. While several embodiments are described in connection with these drawings, there is no intent to limit the disclosure to the embodiment or embodiments disclosed herein. On the contrary, the intent is to cover all alternatives, modifications, and equivalents.

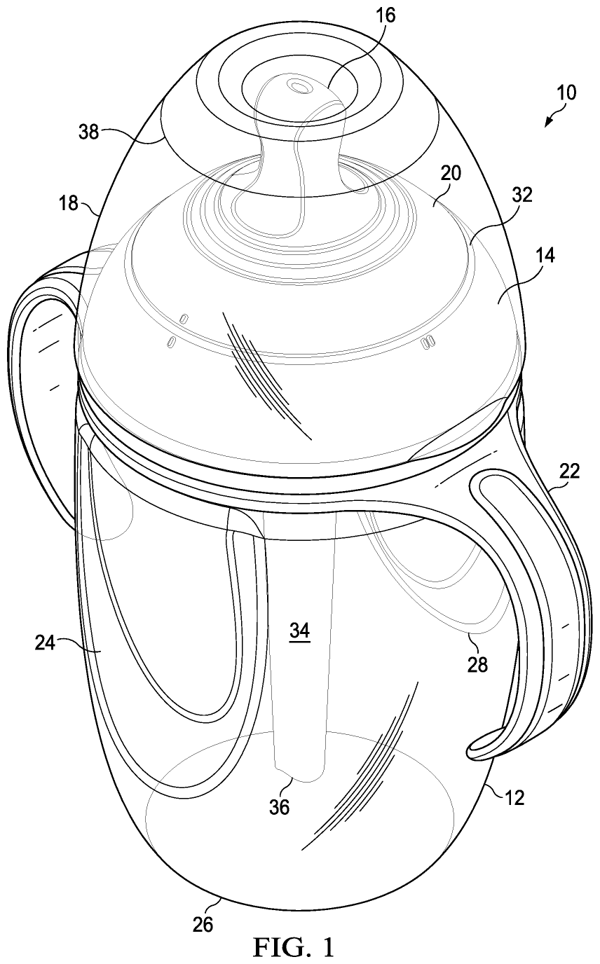

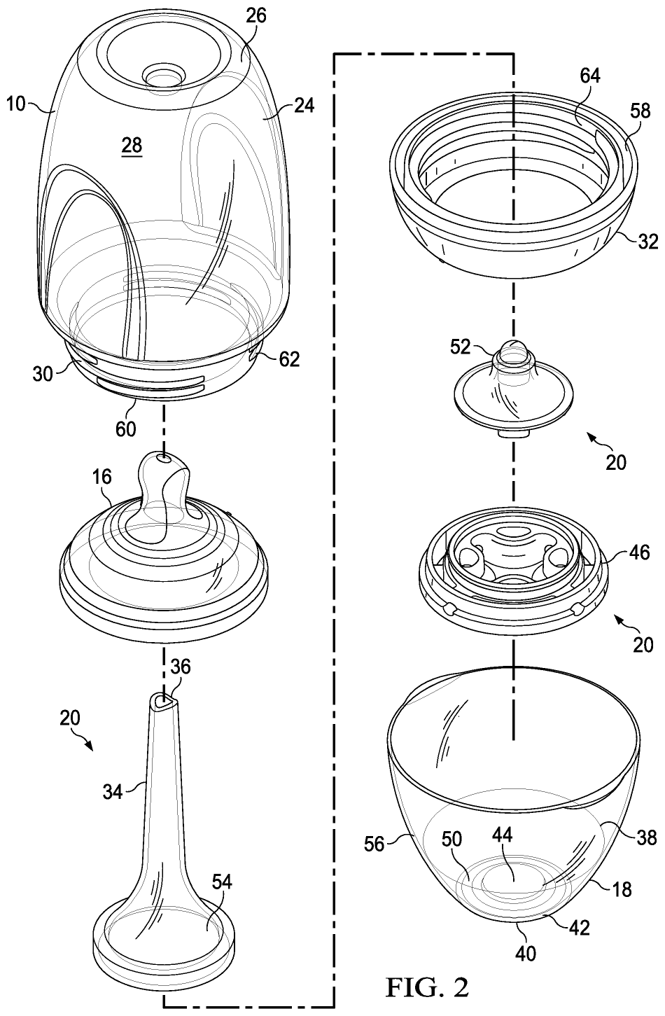

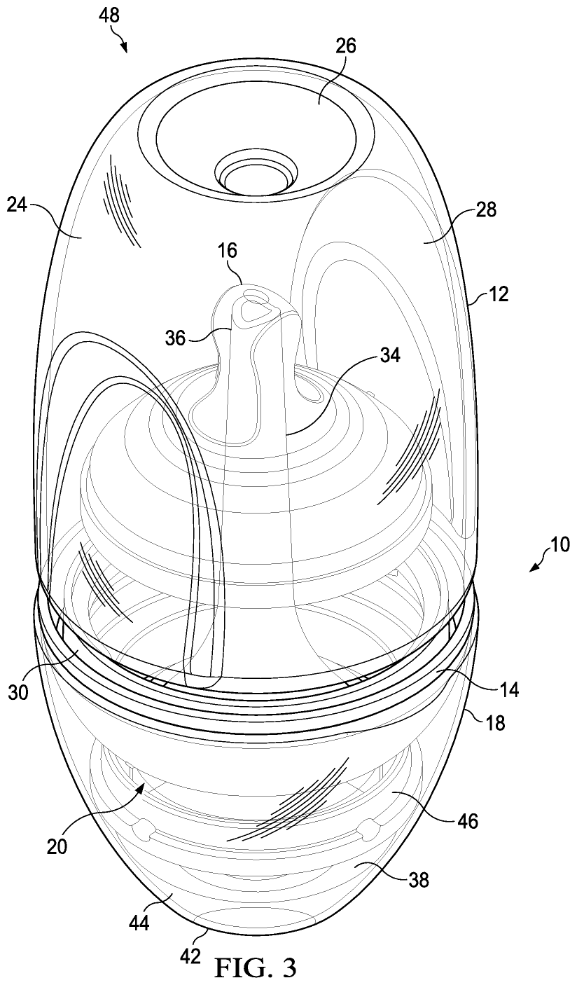

Referring now to FIG. 1, a view of a self-sterilizing bottle illustrating an apparatus useful in the present invention, self-sterilizing bottle 10 is shown in an upright position with container 12, collar 14, mouthpiece 16 which can be either a nipple or spout, cap 18, vent assembly 20 and optional handle 22. Self-sterilizing bottle 10 is shown in an upright position ready for use by user such as a baby or care giver. Container 12 is shown with sidewall 24 and bottom 26 defining a liquid holding space 28. Collar 14 is shown in a first position and fastened to the neck 30 (FIGS. 2 and 3) of container 12. Collar 14 is adapted to receive mouthpiece 16 at mouth 32 and removably fasten mouthpiece 16 to collar 14. Vent assembly 20 is shown with vent straw 34 having distal end 36. Cap 18 is shown with indicia 38 showing a fill line for the cap when in the sterilizing position. In an alternative embodiment not shown, vent assembly 20, including vent straw 34, may be omitted.

Referring now to FIG. 2, an exploded view of the of the self-sterilizing bottle shown in FIG. 1 and oriented for use in the self-sterilization method, cap 18 is shown in the inverted position with flat surface 42 placed on support surface 40 and arranged to hold liquid 44 filed to fill line 50 indicated by indicia 38. Vent assembly 20 is shown comprised of valve body 46, vent valve 52 and vent straw 34. Vent assembly 20 is more fully described in co-pending application serial no. ______, filed contemporaneously herewith for No Colic Bottle and incorporated by reference as if set out in full here. Vent straw 34 has distal end 36 and reservoir end 54. Valve body 46 has a diameter selected so that is rests in cap 18 at a position above indicia 38. In the preferred embodiment, indicia 38 is selected so that when liquid is filled to fill line 50 represented by indicia 38, cap 18 holds 20 ml of water, water being the preferred liquid. If more or less liquid is desired, indicia 38 may be adjusted accordingly with as little as 10 ml of water being sufficient to self-sterilize a bottle. Vent valve 52 is supported by valve body 46 and fits within reservoir end 54 of vent straw 34. The entire vent assembly 20 is placed in cap 18 all supported by valve body 46 resting against cap wall 56. Collar 14 has aperture 58 that is adapted to removably fasten mouthpiece 16 to collar 14 when in nursing mode and to rest against cap wall 56 in sterilization mode. Vent assembly 20 and in particular distal end 36 of vent straw 34 is adapted to support mouthpiece 16 when mouthpiece 16 is positioned in liquid holding space 28 of container 12. Liquid holding space 28 is defined by sidewall 24 and bottom 26. Opposite bottom 26 is neck 30 that further defines opening 60 of container 12. Neck 30 has threads 62 adapted to mate complimentarily with collar threads 64 to seal collar 14 to container 12.

Referring now to FIG. 3, a view of the self-sterilizing bottle shown in FIGS. 1 and 2 adapted for use in the method of the present invention, self-sterilizing bottle 10 is shown in the sterilizing position for self-sterilization mode. Cap 18 is shown in an inverted position resting on a support surface 40. In the preferred embodiment, cap 18 has flat surface 42 for stably supporting cap 18 on support surface 40. Cap 18 is shown with liquid 44, preferably water, up to indicia 38, in preparation for engaging in the self-sterilization method. Valve body 46 of vent assembly 20 is adapted to rest in cap 18 above the liquid fill line indicated by indicia 38. Vent assembly 20, as more fully described in co-pending application serial no. ______, filed contemporaneously herewith for No Colic Bottle and incorporated by reference as if set out in full here, rests in cap 18 above the liquid fill line 50 indicated by indicia 38. Resting on valve body 46 is vent straw 34 with distal end 36 supporting mouthpiece 16. Collar 14 is placed in the second position so that it is loosely fitted to cap 18 along cap wall 56 and supports container 12. In the preferred embodiment, container 12 is not fastened to collar 14 but rests loosely within collar 14. Mouthpiece 16, supported at distal end 36 of vent straw 34, is positioned in liquid holding space 28 defined by sidewall 24 and bottom 26. It should be understood that different orders of assembly may be employed without departing from the scope of the invention.

Using the components shown and described in connection with FIGS. 2 and 3, the self-sterilization method is described. Initially, flat surface 42 of cap 18 is placed on support surface 40 in an inverted position. Liquid 44, such as water, is added to cap 18, preferably to a fill line 50 shown as indicia 38. In the preferred embodiment, indicia 38 is positioned on cap 18 so that the amount of liquid 44 is equal to 20 ml. In one embodiment, after liquid 44 is added to cap 18, collar 14 is placed in cap 18. Separately, mouthpiece 16 is inserted into the liquid holding space 28 of container 12 and container 12 is placed on collar 14. Cap 18, liquid 44, collar 14, mouthpiece 16 and container 12 when assembled in the manner described comprise a self-sterilizing assembly 48. In an alternative embodiment, vent assembly 20 is placed in cap 18 and mouthpiece 16 is supported in liquid holding space 28 by distal end 36 of vent straw 34. In other alternative embodiments, vent assembly 20 may be limited to vent straw 34 or to the combination of vent straw 34 and vent body 46. In an embodiment where only vent straw 34 is used reservoir end 54 may be positioned below indicia 38 and fill line 50 and actually sitting in the liquid. Self-sterilizing assembly 48 is now ready to be placed in a microwave oven (not shown) for the final step in the method. After placed in the microwave oven, self-sterilizing assembly 48 is heated on high, typically for 3 minutes on high in a microwave oven rated at 500 w to 1000 w. Sterilization generally takes place once the liquid temperature reaches 150° F. It should be recognized that sterilization is a matter of gradation with different levels of sterilization occurring for different purposes. For purposes of this invention, sterilization occurs when the liquid in self-sterilizing assembly 48 begins to vaporize.

Although exemplary embodiments have been shown and described, it will be clear to those of ordinary skill in the art that a number of changes, modifications, or alterations to the disclosure as described may be made. All such changes, modifications, and alterations should therefore be seen as within the scope of the disclosure.

What is claimed is:

1. A self-sterilizing bottle comprising:
A container having a sidewall and a bottom defining a liquid holding space, the sidewall forming an open end with a neck;
A mouthpiece;
A collar having a first position and a second position and adapted to fasten to the neck and for removably fastening the mouthpiece to the collar when in the first position, and
A cap configured to have an upright position and an inverted position and when in the upright position adapted to fasten to the collar and cover the mouthpiece when the mouthpiece and cap are attached to the collar, and when in the inverted position to rest stably on a surface, also when in the inverted position, the cap further adapted to hold a liquid and supporting the collar above the liquid when the collar is in the second position.

2. The self-sterilizing bottle of claim 1, further comprising a vent straw.

3. The self-sterilizing bottle of claim 1, further comprising a vent straw having a reservoir end and a distal end and adapted to support a mouthpiece at the distal end when the vent straw is supported by the cap.

4. The self-sterilizing bottle of claim 1, further comprising a vent assembly.

5. The self-sterilizing bottle of claim 1, further comprising a vent assembly wherein the vent assembly has a vent straw with a distal end and adapted to support the mouthpiece at the distal end when the vent assembly is supported by the cap.

6. The self-sterilizing bottle of claim 1, wherein the cap is adapted to hold 20 ml of liquid when in the inverted position.

7. The self-sterilizing bottle of claim 1, wherein the cap has indicia indicating an optimum amount of liquid for self-sterilization.

8. The self-sterilizing bottle of claim 1, wherein the mouthpiece and cap are constructed of a flexible material.

9. The self-sterilizing bottle of claim 1, wherein the collar and container are constructed of a rigid material.

10. A method for self-sterilizing a bottle wherein the bottle comprises a cap, a collar, a mouthpiece and a container having an open end, the method comprising:
Placing the cap on a support surface in a position to hold a liquid;
Adding a liquid to the cap;
Placing the collar on the cap so that the collar remains above the liquid in the cap;
Placing the mouthpiece within the container;
Placing the open end of the container with the mouthpiece therein on the collar to form a self-sterilizing assembly;
Placing the self-sterilizing assembly in a microwave oven; and
Heating the self-sterilizing assembly in a microwave oven for a time sufficient to sterilize the self-sterilizing assembly.

11. The method of claim 10, wherein the liquid of the self-sterilizing assembly is heated to 150° F. and held at or above that temperature for a time sufficient to sterilize the self-sterilizing assembly.

12. The ergonomic bottle of claim 10, wherein the liquid of the self-sterilizing assembly is at least 20 ml.

13. The ergonomic bottle of claim 10, wherein the liquid of the self-sterilizing assembly is water.

14. A method for self-sterilizing a bottle comprising:
Providing a bottle having a container with a closed end and an open end, a collar, a mouthpiece and a cap having an open end and closed end;
Placing the closed end of the cap on a support surface;
Adding a liquid to the cap;
Placing the collar on the cap so that the collar is above the liquid;
Placing the mouthpiece inside the container;
Placing the open end of the container, with the mouthpiece, on the open end of the cap to form a self-sterilizing assembly;
Placing the self-sterilizing assembly in a microwave oven; and
Heating the liquid in the self-sterilizing assembly to a temperature of 150° F. and holding the liquid temperature at or above 150° F. for a time sufficient to sterilize the self-sterilizing assembly.

15. The method of claim 14, wherein the liquid is water.

16. The method of claim 14, wherein the bottle further comprises a vent straw and further comprises the step of supporting the mouthpiece by the vent straw within the container.

17. The method of claim 14, wherein the mouthpiece and cap are formed of a flexible material.

18. The method of claim 14, wherein the cap further comprises indicia indicating an optimum liquid fill line and the method step of adding the liquid to the indicia.

19. The method of claim 14, wherein the container and collar are constructed of a non-flexible material.

20. The method of claim 14, the collar forms a seal with the cap.
